(19) United States
(12) Patent Application Publication
(10) Pub. No.: US 2012/0207847 A1
(43) Pub. Date: Aug. 16, 2012

(54) Cartilage Repair

(75) Inventors: Timothy J. Butler, Pelham, NH (US); Michael Philbrook, Boston, MA (US); Peter K. Jarrett, Lexington, MA (US)

(73) Assignee: GENZYME CORPORATION, Cambridge, MA (US)

(21) Appl. No.: 13/390,899

(22) PCT Filed: Aug. 10, 2010
(86) PCT No.: PCT/US10/44969

§ 371 Date: Apr. 30, 2012

Related U.S. Application Data
(60) Provisional application No. 61/236,631, filed on Aug. 25, 2009.

(51) Int. Cl. A61K 35/32; A61P 19/00

(52) U.S. Cl. 424/549

(57) ABSTRACT

This invention relates to compositions, methods of preparation thereof, and use thereof for cartilage repair.

### TECHNICAL FIELD

This invention relates to compositions, methods of preparation thereof, and use thereof for cartilage repair.

### BACKGROUND

Cartilage damage is common in humans. If untreated, the damage can progressively worsen and can lead to chronic conditions such as osteoarthritis. A number of different therapeutic methods are currently being used to repair damaged cartilage. Exemplary methods include implantation of chondrocytes or mesenchymal stem cells directly or via a cell delivery vehicle into the osteochondral defect, or using growth factors to promote the repair processes (Gao, et al. Clinical Orthopaedics and Related Research 2004, S62-66). Durability of the repair tissue, certainty of the initial optimal growth factor dosage, or knowledge of the interaction among multiple biofactors are important and sometimes problematic (Gao, et al. Clinical Orthopaedics and Related Research 2004, S62-66). There is an ongoing need for a method which exhibits the ability to repair cartilage.

### SUMMARY

This invention is based, at least in part, on the unexpected discoveries that certain compositions can be used to repair cartilage.

In one aspect, the invention features a composition comprising demineralized bone matrix (DBM) and a formulation of a macromer, wherein the macromer comprises at least one water-soluble region, at least one biodegradable region, and at least one reactive polymerizable group.

In another aspect, the invention features a method of repairing a cartilage defect in a subject comprising administering to a subject at a site of the defect an effective amount of a composition, the composition comprising demineralized bone matrix (DBM) and a formulation of a macromer, wherein the macromer comprises at least one water-soluble region, at least one biodegradable region, and at least one reactive polymerizable group.

In some embodiments, the water soluble region can be selected from poly(ethylene glycol), poly(ethylene oxide), poly(vinyl alcohol), poly(vinylpyrrolidone), poly(ethyloxazoline), polysaccharides, proteins, and combinations thereof. In some embodiments, the water soluble region can be poly(ethylene glycol) (PEG).

In some embodiments, the PEG can have an average molecular weight of from about 3,500 Daltons to about 40,000 Daltons. For example, the PEG can have an average molecular weight of about 25,000 Daltons. In other embodiments, the PEG can have an average molecular weight of about 35,000 Daltons. By “about” we mean ±4%.

In some embodiments, one or more reactive polymerizable groups can be selected from ethylenically or acetylenically unsaturated groups, isocyanates, epoxides (oxiranes), sulfhydryls, succinimides, maleimides, amines, imines, amides, carboxylic acids, sulfonic acids and phosphate groups. For example, one or more reactive polymerizable groups can be ethylenically-unsaturated group. In some embodiments, the ethylenically-unsaturated group can be selected from vinyl groups, allyl groups, unsaturated monocarboxylic acids, diacrylates, oligoacrylates, unsaturated dicarboxylic acids, and unsaturated tricarboxylic acids.

In some embodiments, the biodegradable region can comprise at least one carbonate or dioxanone residue linkage. In some embodiments, the carbonate residue linkage can be derived from a cyclic aliphatic carbonate. For example, the carbonate residue linkage can be a poly (trimethylene carbonate) residue.

In some embodiments, the molar ratio of trimethylene carbonate monomers to each PEG can be from about 2:1 to about 20:1. In other embodiments, the molar ratio of trimethylene carbonate monomers to each PEG can be from about 11:1 to about 15:1.

In some embodiments, the biodegradable region can comprise poly(hydroxy acids), poly(lactones), poly(amino acids), poly(anhydrides), poly(orthoesters), or poly(phosphoesters). In some embodiments, the biodegradable region can comprise poly(alpha-hydroxy acids). For example, the biodegradable region can comprise poly(L-lactide).

In some embodiments, the molar ratio of lactide monomers to each PEG can be from about 1:1 to about 8:1. In some embodiments, the molar ratio of lactide monomers to each PEG can be from about 3:1 to about 5:1.

In some embodiments, the biodegradable region can comprise poly(L-lactide) and poly(trimethylene carbonate). In other embodiments, the macromer can comprise poly(L-lactide), poly(trimethylene carbonate), and acrylate endcaps.

In some embodiments, the composition can further comprise an initiator for inducing polymerization, wherein the initiator is selected from (a) a photo initiator; (b) a chemical initiator; and (c) a thermal initiator.

In some embodiments, the initiator can be a photo initiator. For example, the photo initiator can be eosin Y. In some embodiments, the photo initiator is selected from 2,2-dimethoxy-1,2-diphenylethan-1-one (Ciba), (1-hydroxycyclohexyl-phenyl ketone) (Wangs®), phenyl bis(2,4,6-trimethyl benzoyl) phosphine oxide (SignamAldrich), and 2-methyl-1-[4-(methylthio)phenyl]-2-morpholinopropanone-1 (Ivy Fine Chemicals).

In some embodiments, the initiator can be a chemical initiator. For example, the chemical initiator can use redox chemistry. In some embodiments, the chemical initiator can comprise iron (II) and a peroxide. For example, the peroxide can be t-butyl peroxide.

In some embodiments the initiator is a thermal initiator. In some embodiments, thermal initiator is of the peroxide family or of the family of Azo thermal initiators. For example, the Azo thermal initiator can be azobisisobutyronite (AlBN).

In some embodiments, the composition can further comprise a rheology modifier. For example, the rheology modifier can be hyaluronic acid (HA) or carboxymethyl cellulose (CMC).

In some embodiments, the composition can further comprise a pharmaceutically active ingredient. The pharmaceutically active ingredient can be a bone morphogenic protein, a tissue growth factor, an insulin growth factor, an antioxidant, an antibiotic, or a combination of growth factors. In some embodiments, the pharmaceutically active ingredient can be selected from BMP-2, BMP-4, BMP-6, BMP-7, TGF-B, IGF-1, ascorbate, pyruvate, BHT, gentamycin, vancomycin, the combination of TGF-β and BMP-2, and the combination of TGF-β and IGF-1.

In some embodiments, the composition can be in a hydrated form. For example, the composition can be in the form of a putty.

In some embodiments, the composition can comprise from about 60% to about 98% by weight of the formulation of a macromer. In some embodiments, the formulation of a macromer can comprise from about 5% to about 15% by weight of a macromer. In other embodiments, the formulation of a macromer can comprise from about 5% to about 10% by weight of a macromer.

In some embodiments, the composition can comprise from about 2% to about 40% by weight of DBM. In some embodiments, the composition can comprise from about 30% to about 40% by weight of DBM.

In some embodiments, the formulation of a macromer can comprise a biologically compatible liquid. In some embodiments, the biologically compatible liquid can be PBS or water.

In some embodiments, the method of the present invention can further comprise the step of polymerization, in which the polymerization is initiated by a reaction selected from (i) photo polymerization; (ii) chemical free-radical polymerization; and (iii) thermal free-radical polymerization.

In some embodiments, the polymerization can be carried out at the site of cartilaginous tissues. In some embodiments, the polymerization can be carried out prior to administration. In other embodiments, the polymerization can be carried out at the time of manufacture of the composition.

In some embodiments, polymerization is initiated by visible light. In some embodiments, the polymerization is initiated for from about 10 seconds to about 120 seconds. For example, the polymerization is initiated for from about 30 seconds to about 50 seconds.

In some embodiments, polymerization is initiated by long wave ultraviolet light. In some embodiments, the polymerization is initiated for from about 20 seconds to about 60 seconds.

In some embodiments, polymerization is initiated by thermal energy.

In some embodiments, the method of the present invention can further comprise the step of lyophilizing the composition to give a non-hydrated composition. For example, the non-hydrated composition can be in the form of a dry plug.

In some embodiments, the dry plug can comprise from about 85% to about 96% by weight of DBM. In some embodiments, the dry plug can comprise from about 92% to about 96% by weight of DBM.

In some embodiments, the dry plug can comprise from about 1% to about 4% by weight of a polymerized macromer. In some embodiments, the dry plug can comprise from about 2% to about 4% by weight of a polymerized macromer.

In some embodiments, the dry plug can be prepared by the steps comprising: adding DBM to a formulation of a macromer to form a mixture; loading the mixture into a mold; polymerizing the macromer in the mold; and lyophilizing the mixture in the mold.

In some embodiments, the dry plug can be characterized in that the dry plug exhibits a compressive modulus of about 3 MPa.

In some embodiments, the dry plug can be further characterized in that the dry plug exhibits a maximum compressive stress of about 1.5 MPa.

In some embodiments, the subject can be a mammal. In some embodiments, the subject can be a human.

In some embodiments, the site of the defect can be an osteochondral defect in a joint.

As used herein, “the average molecular weight” refers to the weight average molecular weight (Mw) that can be calculated by

Mw=ΣNi²Mi²/ΣNi Mi

where Ni is the number of molecules of molecular weight Mi.

As used herein, a “biologically compatible liquid” is one that is physiologically acceptable and does not cause unacceptable cellular injury. Examples of such liquids are water, buffers, saline, protein solutions, and sugar solutions.

As used herein, a “region” is a block of a macromer differing in subunit composition from neighboring blocks.

As used herein, a “biodegradable” material is one that decomposes under normal in vivo physiological conditions into components that can be metabolized, resolved, or excreted.

As used herein, “putty” is generally firm yet pliable. It does not crumble. It has a malleable consistency that can be shaped by hand, or forced into bone voids or cancellous interstices, cartilage defects, with manual pressure.

As used herein, a “hydrogel” is a substance formed when an organic polymer (natural or synthetic) is cross-linked via covalent, ionic, or hydrogen bonds to create a three-dimensional open-lattice structure which entraps water molecules to form a gel.

As used herein, the term “subject” or “patient,” used interchangeably, refers to any animal, including mammals, preferably mice, rats, other rodents, rabbits, dogs, cats, swine, cattle, sheep, horses, or primates, and most preferably humans.

As used herein, the phrase “an effective amount” refers to the amount of active compound, pharmaceutical agent, or composition that elicits the biological or medicinal response that is being sought in a tissue, system, animal, individual or human by a researcher, veterinarian, medical doctor or other clinician.

As used here, the term “repair” is intended to mean without limitation repair, regeneration, reconstruction, reconstitution or bulking of tissues.

The details of one or more embodiments of the invention are set forth in the accompanying drawings and the description below. Other features, objects, and advantages of the invention will be apparent from the description and drawings, and from the claims.

### DETAILED DESCRIPTION

This invention is based, at least in part, on the unexpected discoveries that certain compositions can be used to repair cartilage.

### Compositions

The compositions described herein include demineralized bone matrix (DBM) and a formulation of a macromer, in which the macromer comprises at least one water-soluble region, at least one biodegradable region, and at least one reactive polymerizable group.

The compositions of the present invention include from about 2% to about 40% by weight of demineralized bone matrix (DBM) (e.g., from about 5% to about 40%, or from about 10% to about 40%, or from about 15% to about 40%, or from about 20% to about 40%). In some embodiments, the compositions can include from about 20% to about 40% by weight of DBM (e.g., from about 25% to about 40%, or from about 30% to about 40%, or from about 35% to about 40%). In other embodiments, the compositions can include from about 30% to about 40% by weight of DBM (e.g., about 32%, or about 35%, or about 38%, or about 40%).

The compositions include from about 60% to about 98% by weight of a formulation of a macromer (e.g., from about 60% to about 95%, or from about 60% to about 90%, or from about 60% to about 88%, or from about 60% to about 85%). In some embodiments, the compositions comprise from about 65% to about 98% by weight of a formulation of a macromer (e.g., from about 70% to about 98%, or from about 75% to about 98%, or from about 80% to about 98%). In some embodiments, the compositions comprise from about 65% to about 95% by weight of a formulation of a macromer (e.g., from about 70% to about 95%, or from about 70% to about 90%, or from about 75% to about 90%, or from about 80% to about 90%). In some embodiments, the formulation of a macromer can include from about 5% to about 20% by weight of a macromer (e.g., from about 5% to about 15%, or from about 5% to about 12%, or from about 5% to about 10%, or from about 5% to about 8%). In other embodiments, the formulation of a macromer can include from about 7% to about 15% by weight of a macromer (e.g., from about 9% to about 15%, or from about 10% to about 15%). In some embodiments, the formulation of a macromer can include from about 5% to about 10% by weight of a macromer (e.g., about 5%, or about 7%, or about 9%, or about 10%).

A formulation of a macromer refers to a macromer in a carrier. In some embodiments, the carrier includes a biologically compatible liquid. The biologically compatible liquid can be phosphate buffered saline solutions (PBS), water, or Lactated Ringer's solution (LRS). Thus, the macromer can be in a solution of a biologically compatible liquid (e.g., PBS or water). In some embodiments, the biologically compatible liquid can be added to a non-hydrated composition to form a hydrated composition before administration.

In some embodiments, a hydrated composition can also be polymerized and lyophilized to give rise to a non-hydrated composition such as a dry plug.

When the compositions of the present invention are in the form of dry plugs, the compositions can include from about 85% to about 96% by weight of demineralized bone matrix (DBM) (e.g., from about 88% to about 96%, or from about 90% to about 96%, or from about 92% to about 96%, or from about 94% to about 96%). In some embodiments, the composition of the present invention can include from about 92% to about 96% by weight of DBM (e.g., from about 92% to about 95%, or from about 92% to about 94%).

The dry plugs can comprise from about 1% to about 4% by weight of a formulation of a polymerized macromer (e.g., from about 1.5% to about 4%, or from about 2% to about 4%, or from about 2.5% to about 4%, or from about 3% to about 4%). In some embodiments, the compositions include from about 2% to about 4% by weight of a formulation of a polymerized macromer (e.g., about 2%, or about 2.5%, or about 3%, or about 3.5% or about 4%).

The macromers of the present invention include at least one water-soluble region linked to at least one biodegradable region. In some embodiments, the macromers contain one water-soluble region linked to one biodegradable region, with one or both ends capped with a polymerizable group. A water soluble region in a macromer is a water soluble group or block that would be water soluble if prepared as an independent molecule rather than being incorporated into the macromer. In some embodiments, the macromers may include a central water-soluble region and outside two biodegradable regions, with one or both ends capped with a polymerizable group. In some embodiments, the central region may be a biodegradable, and the outer regions may be water-soluble. In some embodiments, the macromers may include one or more of the water-soluble regions and biodegradable regions coupled together in a linear or non-linear (e.g., dendritic) fashion.

### Water-Soluble Region

Water-soluble regions or blocks of the macromers can be made predominantly or entirely of synthetic materials. In some embodiments, synthetic materials of controlled compositions and linkages are preferred over natural materials due to more consistent degradation and release properties. Examples of useful synthetic materials include those prepared from poly(ethylene oxide) or poly(ethylene glycol)(i.e., PEG), partially or fully hydrolyzed poly(vinyl alcohol), poly(vinylpyrrolidone), poly(ethyloxazoline), poly(ethylene oxide)-copoly(propylene oxide) block copolymers (e.g., Pluronics™) (poloxamers and meroxapols), and poloxamines. In some embodiments, the water-soluble regions are made from poly(ethylene glycol) (i.e., PEG). In some embodiments, at least 50% of the macromers are formed of synthetic materials (e.g., at least about 55%, at least about 60%, at least about 65%, at least about 70%, or at least about 75%).

The water-soluble regions (e.g., PEG) of the macromers can have an average molecular weight of from about 3,500 Daltons (Da) to about 40,000 Daltons (e.g., from about 3,500 Da to about 35,000 Da, or from about 3,500 Da to about 30,000 Da, or from about 3,500 Da to about 25,000 Da). In some embodiments, the PEG has an average molecular weight of from about 3,500 Da to about 20,000 Da (e.g., from about 3,500 to about 15,000 Da, or from about 3,500 Da to about 10,000 Da, or from about 3,500 Da to about 5,000 Da). For exmaple, the PEG can have an average molecular weight of about 35,000 Da or about 25,000 Da.

The water-soluble regions of the macromers can also be derived from natural materials. Useful natural and modified natural materials include carboxymethyl cellulose, hydroxyalkylated celluloses such as hydroxyethyl cellulose and methylhydroxypropyl cellulose, polypeptides, polynucleotides, polysaccharides or carbohydrates such as Ficoll™, polysucrose, hyaluronic acid and its derivatives, dextran, heparan sulfate, chondroitin sulfate, heparin, or alginate, and proteins such as gelatin, collagen, albumin, or ovalbumin. In some embodiments, the percentage of natural material does not exceed about 50% by weight of the total water-soluble regions.

### Biodegradable Region

Biodegradable regions or blocks are made of “biodegradable” materials that decomposes under normal in vivo physiological conditions into components which may be metabolized, resolved, and/or excreted. In the macromers of the present invention, at least one biodegradable region can be a carbonate or dioxanone linkage. A carbonate is a functional group with the structure —O—C(O)—O—. The carbonate starting material can be cyclic, such as trimethylene carbonate (TMC). After incorporation into the polymerizable macromer, the carbonate will be present at least in part as R—O—C(O)—O—R′, where R and R′ are other components of the macromer. In some embodiments, the carbonates are the cyclic carbonates, which can react with hydroxy-terminated polymers without release of water. Suitable cyclic carbonates include ethylene carbonate (1,3-dioxolan-2-one), propylene carbonate (4-methyl -1,3-dioxolan-2-one), trimethylene carbonate (1,3-dioxan-2-one), and tetramethylene carbonate (1,3-dioxepan-2-one).

The molar ratio of the carbonate residues to each water-soluble region (e.g., PEG) is from about 5:1 to about 25:1 (e.g., from about 5:1 to about 20:1, or from about 5:1 to about 15:1, or from about 5:1 to about 10:1). In some embodiments, the molar ratio of the carbonate residues to each water-soluble region is from about 6:1 to about 20:1 (e.g., from about 8:1 to about 20:1, or from about 10:1 to about 15:1, or from about 11:1 to about 15:1). In other embodiments, the molar ratio of the carbonate residues to each water-soluble region is from about 11:1 to about 15:1.

In some embodiments, the water-soluble region of the macromer may be intrinsically biodegradable.

Biodegradable regions can also be constructed from monomers, polymers and oligomers of hydroxy acids or other biologically degradable polymers (such as ester, peptide, anhydride, orthoester, and phosphoester bonds) that yield materials that are non-toxic or present as normal metabolites in the body. Suitable poly(hydroxy acids) are poly(glycolic acid), poly(DL-lactic acid) and poly(L-lactic acid). Other suitable materials include, polycarbonates such as poly(trimethylene carbonate), poly(amino acids), poly(anhydrides), poly(orthoesters), and poly(phosphoesters). Polylactones such as poly(epsilon-caprolactone), poly(delta-valerolactone), poly(gamma-butyrolactone) and poly (beta-hydroxybutyrate) are also suitable.

The biodegradable regions can be poly(hydroxy acids). For example, the biodegradable regions can be poly(L-lactide). In some embodiments, the molar ratio of lactide monomers to each water-soluble region is from about 1:1 to about 10:1 (e.g., from about 1:1 to about 8:1, or from about 3:1 to about 8:1, or from about 5:1 to about 8:1). In some embodiments, the molar ratio of lactide monomers to each water-soluble region is from about 3:1 to about 8:1 (e.g., from about 3:1 to about 5:1). In some embodiments, the molar ratio of lactide monomers to each water-soluble region is from about 3:1 to about 5:1.

The biodegradable regions or blocks can include both poly(L-lactide) and poly(trimethylene carbonate). A macromer having such biodegradable regions or blocks can modify the time to degradation of the resulting polymerized macromer, for example, hydrogel. The “hydrogel” is formed of polymerized macromers that are biodegradable, and generally are eliminated by the subject within about up to five years. In some embodiments, a macromer containing a lactate moiety as biodegradable region and end group provides a resulting hydrogel with an estimated degradation time in vivo of from about 3 to about 4 months. In some embodiments, a macromer containing a trimethylene carbonate moiety or dioxanone moiety as a biodegradable region provides a resulting hydrogel with an estimated degradation time in vivo of from about 6 to about 12 months. In some embodiments, a polymer containing a caprolactone moiety as biodegradable region provides a resulting hydrogel with an estimated degradation time in vivo of from about 1 to about 2 years. In some embodiments, a macromer without a biodegradable region can provide a resulting hydrogel with an estimated degradation time in vivo of at least about 2 years. Thus, it is one of the advantages of the present invention that by varying the total amount of biodegradable groups, and selecting the ratio between the number of carbonate or ester linkages (which are relatively slow to hydrolyze) and of lower hydroxy acid linkages (especially glycolide or lactide, which hydrolyze relatively rapidly), the degradation time of hydrogels formed from the macromers can be controlled.

### Polymerizable Groups

Polymerizable groups contain a reactive functional group that has the capacity to reacts spontaneously or under the influence of light, heat or other activating conditions or reagents to form additional covalent bonds resulting in macromer interlinking For example, the polymerizable goup can convert a solution of the macromer into hydrogels. Hydrogels are elastic, and further are both elastic and compliant with soft tissue at low polymer concentrations.

Polymerizable groups include groups capable of polymerizing via free radical polymerization and groups capable of polymerizing via cationic or heterolytic polymerization. Suitable groups include, but are not limited to, ethylenically or acetylenically unsaturated groups, isocyanates, epoxides (oxiranes), sulfhydryls, succinimides, maleimides, amines, imines, amides, carboxylic acids, sulfonic acids and phosphate groups.

Ethylenically unsaturated groups include vinyl groups such as vinyl ethers, N-vinyl amides, allyl groups, unsaturated monocarboxylic acids, unsaturated dicarboxylic acids, and unsaturated tricarboxylic acids. Unsaturated monocarboxylic acids include acrylic acid, methacrylic acid and crotonic acid. Unsaturated dicarboxylic acids include maleic, fumaric, itaconic, mesaconic or citraconic acid. Unsaturated tricarboxylic acids include aconitic acid. Polymerizable groups may also be derivatives of such materials, such as acrylamide, N-isopropylacrylamide, hydroxyethylacrylate, hydroxyethylmethacrylate, and analogous vinyl and allyl compounds.

In general, any polymerizable groups that will covalently bond to a second and that can maintain fluidity when exposed to water for enough time to allow deposition and reaction is of use in making a suitable macromer. Due to their excellent stability and slow reactivity in aqueous solutions, ethylenically unsaturated reactive groups are preferred.

The polymerizable groups can be located at one or more ends of a macromer. In some embodiments, the polymerizable groups can be located in the center of a macromer.

Some representative macromer structures described herein are depicted below. PEG, lactate and acrylate units are used solely for purposes of illustration.

### Some Basic Structures:

(CH₂—CH₂—C)ₓ=(PEG)ₓ

(C(O)O—(CH₂)₃—O)_{y} or (O—(CH₂)₃—OC(O))_{y} (depending on direction)=(TMC)_{y}

(CO—CH(CH₃)—O)_{z} or (O—CH(CH₃)—CO)_{z} (depending on direction)=Lactate repeat unit=(LA)_{z}

—CO—CH═CH₂=Acrylate end group=AA

### Segmented PEG/TMC Copolymer:

HO—(O—(CH₂)₃—O—C(O)[(CH₂—CH₂—O)ₓ—(C(O)—O—(CH₂)₃—O)_{y}]ₙ—H or HO—(TMC)_{y}-[(PEG)ₓ-(TMC)_{y}]ₙ—H

### Segmented PEG/TMC/Lactate Terpolymer:

H—(O—CH(CH₃)—C(O))_{z}—(O—(CH₂)₃—O—C(O))_{y}—[(CH₂—CH₂—O)ₓ—(C(O)—O—(CH₂)₃—O)_{y}]ₙ—(CO—CH(CH₃)—O)_{z}—H or HO—(LA)_{z}-(TMC)_{y}-[(PEG)ₓ-(TMC)_{y}]ₙ—(LA)_{z}—H

### Segmented PEG/TMC Macromer (Acrylated):

CH₂═CH—C(O)—(O—CH₂)₃—O—C(O))_{y}[(CH₂—CH₂—O)ₓ—(C(O)—(CH₂)₃—O)_{y}]ₙ—C(O)—CH═CH₂ or AA-(TMC)_{y}-[(PEG)ₓ-(TMC)_{y}]ₙ-AA

### Segmented PEG/TMC/Lactate Terpolymer Macromer (Acrylated):

AA-(LA)_{z}-(TMC)_{y}-[(PEGₓ-(TMC)_{y}]ₙ-(LA)_{z}-AA

In some embodiments, the macromers include a core of a hydrophilic poly(ethyleneglycol) (PEG) with a molecular weight between about 3,500 Da and 40,000 Da, (e.g., 25,000 Da or 35,000 Da); an extension on both ends of the core which includes 1 to 10 carbonate residues and optionally between one and five hydroxyacid residues, for example, alpha-hydroxy acid residues (e.g., lactic acid residues); wherein the total of all residues in the extensions is sufficiently small to preserve water-solubility of the macromers, being typically less than about 20% of the weight of the macromers, more preferably 10% or less. The ends are capped with ethylenically-unsaturated (i.e., containing carbon-carbon double bonds) caps, with a preferred molecular weight between about 50 and 300 Da, most preferably acrylate groups having a molecular weight of 55 Da. These materials are described in U.S. Pat. No. 6,177,095 to Sawhney, et al. (incorporated herein by reference in its entirety). See also U.S. Pat. No. 5,900,245 to Sawhney, et al. (incorporated herein by reference in its entirety).

In some embodiments, the compositions include a macromer that is a “FocalSeal™”, i.e., a biodegradable, polymerizable macromer having a solubility of at least about 1 g/100 ml in an aqueous solution comprising at least one water soluble region, at least one degradable region which is hydrolyzable under in vivo conditions, and free radical polymerizable end groups having the capacity to form additional covalent bonds resulting in macromer interlinking, wherein the polymerizable end groups are separated from each other by at least one degradable region. Exemplary FocalSeal™ compositions and hydrogels are described in U.S. Pat. No. 5,410,016, U.S. Pat. No. 6,083,524, and U.S. Pat. No. 7,022,343, all of which incorporated herein by reference in their entirety. FocalSeal™ are available from Genzyme Corporation and are provided in a plurality of grades including FOCALSEAL™-S, FOCALSEAL™-L, and FOCALSEAL™-M. All consist of a core of PEG, partially concatenated with monomers which are linked by biodegradable linkages, and capped at each end with a photopolymerizable acrylate group. These differ based on the molecular weight of the core PEG, the number of PEG molecules, and the number and composition of the biodegradable monomers. FOCALSEAL™-S includes PEG with molecular weight 19,400±4000 Daltons; FOCALSEAL™-L and FOCALSEAL™-M include PEG with molecular weight 35,000±5000 Daltons. FOCALSEAL™-S includes trimethylene carbonate monomers in a ratio of at least six or seven TMC molecules to each PEG, typically twelve to thirteen TMC molecules to each PEG, and lactide monomers, typically four lactide molecules to each PEG molecule, with a maximum of five lactide monomers to each PEG. The ratio of TMC molecules:lactate molecules for FOCALSEAL™-S is about 12:4 or 3:1. FOCALSEAL™-M is the same as FOCALSEAL™-S with the exception of the molecular weight of the PEG. FOCALSEAL™-L includes TMC molecules in a ratio of less than ten, more typically seven, TMC molecules to each PEG. U.S. Pat. No. 6,083,524 describes the synthesis in detail of these materials.

In some embodiments, the composition includes a macromer that is commercially available FocalSeal-L. In some embodiments, the composition includes a macromer that is commercially available FocalSeal-S. In other embodiments, one or more commercially available FocalSeal products is blended with another (e.g., FocalSeal-L blended with FocalSeal-S) to provide a desired mix of properties (e.g., half life and stiffness).

In some embodiments, The composition can further comprise a pharmaceutically active ingredient. The pharmaceutically active ingredient can be a bone morphogenic protein, a tissue growth factor, an insulin growth factor, an antioxidant, an antibiotic, or a combination of growth factors. In embodiments, the pharmaceutically active ingredient can be selected from BMP-2, BMP-4, BMP-6, BMP-7, TGF-B, IGF-1, ascorbate, pyruvate, BHT, gentamycin, vancomycin, the combination of TGF-13 and BMP-2, and the combination of TGF-β and IGF-1.

In some embodiments, a composition described herein is blended with another agent that can be used for tissue augmentation and/or repair such as a gel of hyaluronic acid such as hylan B, or collagen.

Other compounds that can be added to the macromer containing compositions include, but are not limited to, a drug to manage pain, such as lidocain, antiinflammatory drugs, steroids, chemotherapueutics, or Botulinum Toxin. Stabilizers which prevent premature polymerization can be included, for example, quinones, hydroquinones, or hindered phenols.

### Preparation of Compositions

Demineralized bone matrix (DBM) is the protein component of bone. It can be prepared using the methods well known to those skilled in the art. General synthetic methods are found in the literature. See Yee et al. Spine (2003), 28 (21) and Colnot et al. Clinical Orthopaedics and Related Research (2005), 435, 69-78. For example, demineralized bone matrix (DBM) can be prepared by acid extraction of allograft bone, resulting in loss of most of the mineralized component but retention of collagen and non-collagen proteins, including growth factors. DBM can be processed as crushed granules, powder or chips. It can be formulated for use as granules, gels, sponge material or putty and can be freeze-dried for storage. Additionally, DBM can be obtained from sources such as Tissue Banks International (TBI), San Rafael, Calif. or Exactech, Gainesville, Fla.

The compositions of the present invention can be prepared by adding demineralized bone matrix (DBM) to a macromer solution, for example, a macromer in a solution of biologically compatible liquid (e.g., PBS or water). Alternatively, the compositions of the present invention can be prepared by adding a biologically compatible liquid to a dry mixture of DBM and a macromer. In some embodiments, a photo initiator, or a chemical initiator, or a thermal initiator can be added to the compositions.

In some embodiments, the compositions including DBM and a formulation of a macromer can form a viscous and cohesive mass that results in an injectable and moldable putty. A desirable putty should not show any sign of “dry edge” when pressure is applied to squeeze out the ball shaped putty. The composition may be stored at about −40° C. and sealed from the light to maintain its stability and prevent shelf-degradation of the putty. When used in surgery, the putty can convert to a semisolid mass after initiation of polymerization (e.g., photo-polymerization). In cases when photo-polymerization is initiated, the rate of crosslinking reaction depends on the light intensity and the duration of the exposure. In some embodiments, exposure to the operating room light can be sufficient to cause the macromer some degree of cross-linking

After polymerization, the resulting compositions can be loaded into a mold. The mold can be made of Teflon. The loaded compositions can be lyophilized to give a dry plug. A dry plug is a porous, osteoconductive structure. It is a dry formulation of DBM and a macromer and therefore will have enhanced stability at room temperature.

Alternatively, prior to polymerization, the compositions can be loaded into a mold and polymerized. The polymerized compositions in the mold can then be lyophilized to give dry plugs.

In some embodiments, the dry plug includes demineralized bone matrix (DBM) and crosslinked FocalSeal-S. The dry plug can be prepared, for example, by adding DBM to a 1% solution of FocalSeal-S with a 0.1% concentration of vinylcaprolactam (VC). The DBM and FocalSeal-S mixture can be then loaded into a Teflon mold, photo crosslinked and then lyophilized to give a dry plug. The obtained dry plug can include about 2.2% (w/w) FocalSeal-S, 94.6% (w/w) DBM, and 3.2% (w/w) salts and VC. The prepared dry plug can have the following physical properties:

TABLE 1
Physical Properties of a Dry Plug

| | |
|---|---|
| % Linear, confined swelling | about 15-20% |
| Modulus | about 3 MPa |
| Compressive Stress (10% strain) | about 0.2 MPa |
| Maximum Compressive Stress | about 1.5 MPa |

The macromers described herein can be synthesized using means well known to those of skill in the art. General synthetic methods are found in the literature, for example in U.S. Pat. No. 5,410,016 (Hubbell et al.), U.S. Pat. No. 4,243,775 (Rosensaft et al.), and U.S. Pat. No. 4,526,938 (Churchill et al.) (incorporated herein by reference in their entirety). For example, a polyethylene glycol backbone can be reacted with trimethylene carbonate (TMC) or a similar carbonate to form a TMC-PEG polymer. The TMC-PEG polymer may optionally be further derivatized with additional degradable groups, such as lactate groups (see Jarrett et al. U.S. Pat. No. 6,083,524). The terminal hydroxyl groups can then be reacted with acryloyl chloride in the presence of a tertiary amine to end-cap the polymer with acrylate end-groups. Similar coupling chemistry can be employed for macromers containing other water-soluble blocks, biodegradable blocks, and polymerizable groups, particularly those containing hydroxyl groups.

When polyethylene glycol is reacted with TMC and a cyclic ester of a hydroxy acid such as glycolide or lactide, the reaction can be either simultaneous or sequential. The simultaneous reaction will produce an at least partially random copolymer of the three components. Sequential addition of a lactide after reaction of the PEG with the TMC will tend to produce an inner copolymer of TMC and one or more PEGs, which will statistically contain more than one PEG residue linked by linkages derived from TMC, with hydroxy acid moieties largely at the ends of the (TMC, PEG) region.

### Polymerization

The compositions of the present invention may be polymerized into a pre-selected shape at a site remote from the surgery room (e.g., at a site of manufacture of the compositions). For example, a dry plug can be prepared by polymerizing a macromer in a mold that is loaded with a mixture of DBM and a formulation of macromer, followed by lyophilizing the macromer in the mold. In some embodiments, the dry plug can be characterized in that the dry plug can exhibit a maximum compressive stress of about 1.5 MPa.

The compositions can also be polymerized prior to administration in the surgery room. In some embodiments, the compositions can be polymerized at the site of cartilaginous tissues in the body.

The macromer in a composition can be polymerized by either free radical (homolytic) processes or by heterolytic processes (such as cationic polymerization). In some embodiments, the macromer can be polymerizable by free radical polymerization. Polymerizable groups for free radical polymerization can be acrylates, diacrylates, oligoacrylates, methacrylates, dimethacrylates, oligomethacrylates, cinnamates, dicinnamates, oligocinnamates.

Polymerization can be initiated by any convenient reaction, including photopolymerization, chemical or thermal free-radical polymerization, redox reactions, cationic polymerization, and chemical reaction of active groups (e.g., isocyanates). In some embodiments, polymerization can be initiated using initiators. The term “initiator” is used herein in a broad sense, in that it is a composition which under appropriate conditions will result in the polymerization of macromers. Materials for initiation may be photo initiators, chemical initiators, thermal initiators, photosensitizers, co-catalysts, chain transfer agents, and radical transfer agents. All initiators known in the art are potentially suitable for the practice of the priming technique. The critical property of an initiator is that the polymerization will not proceed at a useful rate without the presence of the initiator.

Photo initiators can generate a free radical on exposure to light, including UV (ultraviolet) and IR (infrared) light. In some embodiments, polymerization is initiated by long-wavelength ultraviolet light (LWUV) or visible light, for example, 320 nm or higher, for example, between about 365 and about 550 nm. LWUV and visible light are preferred because they cause less damage to tissue and other biological materials than short-wave UV light.

Suitable photo initiators are those which can initiate polymerization of the macromers without cytotoxicity and within a short time frame, minutes at most and most preferably seconds. Such photo initiators include, but are not limited to, erythrosin, phloxime, rose bengal, thionine, camphorquinone, ethyl eosin, eosin, methylene blue, riboflavin, 2,2-dimethyl-2-phenylacetophenone, 2,2-dimethoxy-2-phenyl acetophenone 2-methoxy-2-phenylacetophenone, or 2,2-dimethoxy-1,2-diphenylethan-l-one known as Irgacure 651 (available form Ciba Specialty Chemicals), or any other photo initiators from the Irgacure family. In some embodiments, the photo initiator is Eosin Y. In some embodiments, the photo initiator is of the Irgacure family. For example, the photo initiator can be selected from Irgacure 651 (2,2-dimethoxy-1,2-diphenylethan-l-one), Irgacure 184 (1-hydroxycyclohexyl-phenyl ketone), Irgacure 819 (phenyl bis(2,4,6-trimethyl benzoyl) phosphine oxide), and Irgacure 907 (2-methyl-1-[4-(methylthio)phenyl]-2-morpholinopropanone-1). In some embodiments, the macromer requires from about 1-3% by weight of the photo initiator.

Another alternative class of initiators capable of initiating polymerization of free radically active functional groups includes conventional chemical initiator systems such as redox system. The redox system may include, but are not limited to, iron (II) (e.g., ferrous gluconate) and a peroxide (e.g., t-butyl peroxide or hydrogen peroxide). In some cases, it is advantageous to use a redox system for polymerization, because the associated free radical initiation may be triggered at a reasonable rate over a wide range of temperatures, and may even be triggered at low temperature of between 0-20° C.

Examples of suitable thermal initiators include, but are not limited to, 2,2′-azobis (2,4-dimethylpentanenitrile), 2,2′-azobis (2-methylpropanenitrile), 2,2′-azobis (2-methylbutanenitrile), peroxides such as benzoyl peroxide, and the like. Preferably, the thermal initiator is azobisisobutyronite (AlBN). Other well-known azo-compounds are also useful.

To facilitate the administration and treatment of patients with compositions described herein, the macromer can be polymerized in the presence of pharmaceutically active ingredient, such as prophylactic, therapeutic or diagnostic agents, for delivery of the incorporated agents in a controlled manner as the resulting polymer degrades. The pharmaceutically active ingredient can be a bone morphogenic protein, a tissue growth factor, an insulin growth factor, an antioxidant, an antibiotic, or a combination of growth factors. In some embodiments, the pharmaceutically active ingredient can be selected from BMP-2, BMP-4, BMP-6, BMP-7, TGF-B, IGF-1, ascorbate, pyruvate, BHT, gentamycin, vancomycin, the combination of TGF-β and BMP-2, and the combination of TGF-β and IGF-1.

In some embodiments, pharmaceutically active agents that may be coadministered with the compositions can be anesthetics (such as lidocaine) and antiinflammatories (such as cortisone).

The macromers described herein generally have tailorable properties such as solubility and solution viscosity properties. For a given solution concentration in water, the viscosity is generally affected by the degree of end linking, the length of the TMC (and other hydrophobic species) segments, and the molecular weight of the starting water-soluble regions (e.g., PEG). The modulus of the hydrogel is affected by the molecular weight between crosslinks. The hydrogel degradation rate can be modified, for example, by adding a second, more easily hydrolyzed polymerization region (e.g. lactate, glycolate, 1,4-dioxanone) as a segment on the ends of the basic PEG/TMC copolymer prior to adding the crosslinkable end group to form the macromer.

In some cases it is desirable to increase the viscosity of the macromer at the time of application to the tissue so that the macromer remains more firmly at the site of application. Polymers which can be used to increase the viscosity of the macromer solution include, but are not limited to, glycosaminoglycans (GAG) such as hyaluronic acid (HA), carboxymethyl cellulose (CMC), dextran, dextran sulfate, and polyvinylpyrrolidone (PVP). These can be added to the macromer solution immediately before application to the tissue.

### Method of Use

The compositions of the present invention can be used to repair cartilage in a subject. The compositions can be administered to the subject at a site of a defect in cartilaginous tissue or a combination of bone and cartilage defect such as in an osteochondral defects. The compositions of the present invention can also be used to repair bone or a defect in other tissues such as meniscus, ligament, tendon, and intervertebral disc annulus. Effective doses will depend on the disease condition being treated as well as by the judgment of the attending clinician depending upon factors such as the severity of the disease, the age, weight and general condition of the patient, and the like.

The compositions of the invention may be applied directly to the tissue and/or to the site in need of cartilage repair. In some embodiments, the site of treatment in the body may be surgically prepared to remove abnormal tissues, followed by placing the composition of the present invention in the defect area. Alternatively, surgical preparation includes piercing, abrading or drilling into adjacent tissue regions or vascularized regions to create channels for the cells or bone marrow to migrate into the plug or putty. The compositions of the invention can be used to fill an osteochondral defect, or a defect that includes microfractures, or a chondral defect.

The compositions can be administered with a syringe and needle or a variety of devices. Several delivery devices have been developed and described in the art to administer viscous liquids such as the carpule devices described by Dr. Orentriech in U.S. Pat. Nos. 4,664,655 and 4,758,234 which are hereby incorporated by reference in their entirety. Additionally, to make delivery of the compositions as easy as possible for the doctors, a leveraged injection ratchet mechanism or powered delivery mechanism may be used. It is currently preferred for the compositions to be preloaded in a cylindrical container or cartridge having two ends. The first end would be adapted to receive a plunger and would have a movable seal placed therein. The second end or outlet would be covered by a removable seal and be adapted to fit into a needle housing to allow the compositions in the container to exit the outlet and enter a needle or other hollow tubular member of the administration device. It is also envisioned that the compositions could be sold in the form of a kit comprising a device containing the composition. The device having an outlet for said composition, an ejector for expelling the composition and a hollow tubular member fitted to the outlet for administering the composition into an animal.

Once the compositions are administered to the subject, the compositions can be polymerized, for example, by irradiating the compositions. The subject can be subjected to a illuminating light, which initiates polymerization of the administered compositions. When polymerization is achieved using radiation, the subject is generally administered radiation by illumination for at least from about 10 seconds to about 120 seconds (e.g., at least about 10 seconds, at least about 15 seconds, at least about 20 seconds, at least about 25 seconds, at least about 30 seconds, at least about 35 seconds, at least about 45 seconds, at least about 60 seconds, at least about 90 seconds, or at least about 120 seconds). In some embodiments, when polymerization can be achieved using radiation, the subject can be administered radiation by illumination for at least about 30 seconds to about 50 seconds (e.g., at least about 30 seconds, at least about 35 seconds, at least about 40 seconds, at least about 45 seconds, or at least about 50 seconds). When polymerization is carried out by irradiating a subject with long wave ultraviolet light, the irradiating can take from at least from about 20 seconds to about 60 seconds (e.g., at least about 20 seconds, at least about 25 seconds, at least about 30 seconds, at least about30 seconds, at least about 35 seconds, at least about 40 seconds, at least about 45 seconds, at least about 45 seconds, at least about 50 seconds, at least about 55 seconds, or at least about 60 seconds).

The compositions can also be administered to a subject in an iterative manner, such that at least two, for example, 3, 4, or 5 applications of the composition are provided to the subject, where the compositions are polymerized between each new administration of the compositions.

The compositions described herein can be packaged in any convenient way, and may form a kit including for example separate containers, alone or together with the application device. The macromers are preferably stored separately from the initiator, unless they are co-lyophilized and stored in the dark, or otherwise maintained unreactive. Dilute initiator can be in the reconstitution fluid; stabilizers are in the macromer or syringe; and other ingredients may be in either vial, depending on chemical compatibility. The DBM may be included in the kit as a powder to be reconstituted with a physiologically acceptable fluid prior to mixing such as the initiator solution or the mixed macromer/initator solution. If a drug is to be delivered in the composition, it may be in any of the vials, or in a separate container, depending on its stability and storage requirements.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Various modifications of the invention, in addition to those described herein, will be apparent to those skilled in the art from the foregoing description. Such modifications are also intended to fall within the scope of the appended claims.

1. A method of repairing a cartilage defect in a subject comprising administering to a subject at a site of the defect an effective amount of a composition, the composition comprising demineralized bone matrix (DBM) and a formulation of a macromer, wherein the macromer comprises at least one water-soluble region, at least one biodegradable region, and at least one reactive polymerizable group.

2. The method of claim 1, wherein said water soluble region is selected from poly(ethylene glycol), poly(ethylene oxide), poly(vinyl alcohol), poly(vinylpyrrolidone), poly(ethyloxazoline), polysaccharides, proteins, and combinations thereof.

3. The method of claim 1, wherein said water soluble region is poly(ethylene glycol) (PEG).

4-6. (canceled)

7. The method of claim 1, wherein said one or more reactive polymerizable groups are selected from ethylenically or acetylenically unsaturated groups, isocyanates, epoxides (oxiranes), sulfhydryls, succinimides, maleimides, amines, imines, amides, carboxylic acids, sulfonic acids and phosphate groups.

8. (canceled)

9. The method of claim 7, wherein the ethylenically-unsaturated group is selected from vinyl groups, allyl groups, unsaturated monocarboxylic acids, diacrylates, oligoacrylates, unsaturated dicarboxylic acids, and unsaturated tricarboxylic acids.

10. The method of claim 1, wherein the biodegradable region comprises at least one carbonate or dioxanone residue linkage.

11. The method of claim 10, wherein the carbonate residue linkage is derived from a cyclic aliphatic carbonate.

12. The method of claim 10, wherein the carbonate residue linkage is a poly (trimethylene carbonate) residue.

13-14. (canceled)

15. The method of claim 1, wherein the biodegradable region comprises poly(hydroxy acids), poly(lactones), poly(amino acids), poly(anhydrides), poly(orthoesters), or poly(phosphoesters).

16. (canceled)

17. The method of claim 1, wherein the biodegradable region comprises poly(L-lactide).

18-19. (canceled)

20. The method of claim 1, wherein the biodegradable region comprises poly(L-lactide) and poly(trimethylene carbonate).

21. The method of claim 1, wherein the macromer comprises poly(L-lactide), poly(trimethylene carbonate), and acrylate endcaps.

22. The method of claim 1, wherein the composition further comprises an initiator for inducing polymerization, wherein the initiator is selected from (a) a photo initiator; (b) a chemical initiator; and (c) a thermal initiator.

23-28. (canceled)

29. The method of claim 22, wherein the initiator is a thermal initiator.

30-31. (canceled)

32. The method of claim 1, wherein the composition further comprises a rheology modifier.

33. (canceled)

34. The method of claim 1, wherein the composition further comprises a pharmaceutically active ingredient.

35-36. (canceled)

37. The method of claim 1, wherein the composition is in a hydrated form.

38. The method of claim 37, wherein said composition is in the form of a putty.

39-45. (canceled)

46. The method of claim 37 further comprising the step of polymerization, wherein the polymerization is initiated by a reaction selected from (i) photo polymerization; (ii) chemical free-radical polymerization; and (iii) thermal free-radical polymerization.

47. The method of claim 46, wherein said polymerization is carried out at the site of cartilaginous tissues.

48-55. (canceled)

56. The method of claim 46 further comprising the step of lyophilizing the composition to give a non-hydrated composition.

57. The method of claim 56, wherein the non-hydrate composition is in the form of a dry plug.

58. The method of claim 57, wherein the dry plug comprises from about 85% to about 96% by weight of DBM.

59. The method of claim 57, wherein the dry plug comprises from about 92% to about 96% by weight of DBM.

60. The method of claim 57, wherein the dry plug comprises from about 1% to about 4% by weight of a polymerized macromer.

61. (canceled)

62. The method of claim 57, wherein the dry plug is characterized in that the dry plug exhibits a compressive modulus of about 3 MPa.

63. The method of claim 62, wherein the dry plug is further characterized in that the dry plug exhibits a maximum compressive stress of about 1.5 MPa.

64. The method of claim 1, wherein the subject is a mammal.

65. The method of claim 1, wherein the subject is a human.

66. The method of claim 1, wherein the site of the defect is an osteochondral defect in a joint.
